(19) United States
(12) Patent Application Publication
(10) Pub. No.: US 2025/0295327 A1
(43) Pub. Date: Sep. 25, 2025

(54) COUGH DETECTION IN A GARMENT

(71) Applicant: HILL-ROM SERVICES PTE. LTD., Singapore (SG)

(72) Inventors: Aaron Alexander AYU, Singapore (SG); Yaolong LOU, Singapore (SG); Bobby Png Gee HAN, Singapore (SG); Karen MULLERY, St. Paul, MN (US); Stacey REULAND, St. Paul, MN (US); Barry Tay Eng KEONG, Singapore (SG); GuoXiang WANG, Singapore (SG)

(21) Appl. No.: 19/086,821

(22) Filed: Mar. 21, 2025

Related U.S. Application Data
(60) Provisional application No. 63/568,765, filed on Mar. 22, 2024.

(51) Int. Cl. A61B 5/08; A61B 5/00; A61H 9/00; A61H 23/04; A61H 31/00

(52) U.S. Cl. CPC A61B 5/0823; A61B 5/6805; A61B 5/725; A61B 5/7282; A61H 9/0078; A61H 23/04; A61H 31/00; A61H 2201/165; A61H 2230/405

(57) ABSTRACT

The present disclosure relates to the use of sensors in chest compression devices to automatically detect an oncoming cough when a patient is using such a device for airway clearance. The chest compression device is configured to pause when the oncoming cough is detected based on one or more cough indicators.

### CROSS-REFERENCE TO RELATED APPLICATIONS

This application claims priority under 35 U.S.C. § 119(e) to U.S. Provisional Application No. 63/568,765, filed Mar. 22, 2024, which is expressly incorporated by reference herein.

### BACKGROUND

The present disclosure relates to the use of sensors in chest compression devices to automatically detect an oncoming cough when a patient is using such a device for airway clearance.

Manual percussion techniques of chest physiotherapy have been used for a variety of diseases, such as cystic fibrosis, emphysema, asthma, and chronic bronchitis, to remove excess mucus that collects in the lungs. To bypass dependency on a caregiver to provide this therapy, chest compression devices have been developed to produce high-frequency chest wall oscillation (HFCWO), a very successful method of airway clearance.

A typical HFCWO system includes an inflatable garment that is attached to an air plethysmograph device or an air pulse generator through one or more air hoses. The HFCWO system mechanically performs chest physical therapy by vibrating at a high frequency. The garment vibrates the chest to loosen and thin mucus in the respiratory system. However, such HFCWO systems typically require the patient to stop the machine intermittently when they need to cough.

These HFCWO systems may be used in a patient's home or in hospitals. However, successful use in the home is dependent on regular use of the device by the patient. Ease of use is an important factor in gaining acceptable patient compliance. Accordingly, there is a need in the healthcare field to have HFCWO systems that are easy to use and have an ergonomic operation. There is a need for HFCWO systems that are not dependent on patient action for pausing and that can pause automatically when the patient has to cough.

### SUMMARY

The present disclosure includes one or more of the features recited in the appended claims and/or the following features which, alone or in any combination, may comprise patentable subject matter.

According to a first aspect of the present disclosure, a high-frequency chest wall oscillation therapy apparatus comprises a garment including a number of air sacs; an air pulse generator including an air chamber fluidly connected to the garment and a sensor, wherein rapid mechanical compression of air in the air chamber causes the inflation of the sacs, and wherein the sensor is positioned in pneumatic communication with the air chamber and configured to measure pressure in the air chamber or the garment, and a controller coupled to the sensor, the controller including a processor and a memory device, the memory device including instructions that, when executed by the processor, cause the controller to receive a signal from the sensor to measure the pressure in the air chamber or the garment over time, evaluate the signal to determine a cough indicator and generate an output command to modify a therapy provided to a patient wearing the garment in response to the cough indicator.

In some embodiments of the first aspect, the controller is configured to receive a first signal from the sensor when the patient is inhaling and a second signal from the sensor when the patient is exhaling, analyze the first and the second signals to determine the cough indicator.

In some embodiments of the first aspect, the controller is configured to generate the output command to indicate an oncoming cough if the cough indicator exceeds a threshold range by a threshold percentage.

In some embodiments of the first aspect, the garment is configured to vibrate a patient chest when inflated, and the output command is configured to automatically pause the vibration of the garment when the cough indicator exceeds the threshold range by the threshold percentage.

In some embodiments of the first aspect, the air pulse generator is fluidly connected to the garment with one or more hoses, and wherein the air pulse generator, the hoses, and the garment form a closed system.

In some embodiments of the first aspect, the cough indicator is a rate of change of pressure.

In some embodiments of the first aspect, the controller is configured to process the signal with a low pass filter and then differentiate the signal to calculate the rate of change of pressure.

In some embodiments of the first aspect, the controller is configured to calculate the rate of change of pressure for a fixed time interval to determine the threshold range.

In some embodiments of the first aspect, the fixed time interval is about 30 seconds.

In some embodiments of the first aspect, threshold percentage is about 40%, and if the rate of change of pressure exceeds the threshold range by about 40%, the output command indicates an oncoming cough.

In some embodiments of the first aspect, the cough indicator is pressure acceleration.

In some embodiments of the first aspect, the controller is configured to process the signal with a low pass filter and then double differentiate the signal to calculate the pressure acceleration.

In some embodiments of the first aspect, the controller is configured to calculate the pressure acceleration for a fixed time interval to determine the threshold range.

In some embodiments of the first aspect, the fixed time interval is about 30 seconds.

In some embodiments of the first aspect, the threshold percentage is about 40%, and if the pressure acceleration exceeds the threshold range by about 40%, the output command indicates an oncoming cough.

In some embodiments of the first aspect, the air pulse generator is configured to automatically pause to allow the patient to cough if the output command is indicative of an oncoming cough.

According to a second aspect of the present disclosure, a method of diagnosing an oncoming cough in a patient using a high-frequency chest wall oscillation therapy apparatus, the method comprises wearing a garment including a number of air sacs; inflating the garment by using an air pulse generator including an air chamber fluidly connected to the garment; receiving a signal indicative of pressure in the garment and the air chamber over a period of time by using a sensor positioned in the air pulse generator; processing the signal over a period of time to determine a cough indicator; diagnosing the oncoming cough if the cough indicator exceeds a threshold value by a threshold percentage; generating an output command to indicate the oncoming cough; and modifying therapy to a patient wearing the garment in response to the cough indicator.

In some embodiments of the second aspect, the method further comprises receiving a first signal indicative of a first pressure when inhaling air and receiving a second signal indicative of a second pressure when expelling air.

In some embodiments of the second aspect, the method further comprises the garment vibrating the patient chest after inflating the garment.

In some embodiments of the second aspect, the method further comprises generating the output command to automatically pause the vibration of the garment when the cough indicator exceeds a threshold value by a threshold percentage.

In some embodiments of the second aspect, the cough indicator is a rate of change of pressure.

In some embodiments of the second aspect, the method further comprises processing the signal with a low pass filter and then differentiating the signal to calculate the rate of change of pressure.

In some embodiments of the second aspect, the method further comprises calculating the rate of change of pressure for a fixed time interval to determine the threshold range.

In some embodiments of the second aspect, the fixed time interval is about 30 seconds.

In some embodiments of the second aspect, the threshold percentage is about 40%, and if the rate of change of pressure exceeds the threshold range by about 40%, the output command indicates an oncoming cough.

In some embodiments of the second aspect, the cough indicator is pressure acceleration.

In some embodiments of the second aspect, the method further comprises processing the signal with a low pass filter and then double differentiating the signal to calculate the pressure acceleration.

In some embodiments of the second aspect, the method further comprises calculating the pressure acceleration for a fixed time interval to determine the threshold range.

In some embodiments of the second aspect, the fixed time interval is about 30 seconds.

In some embodiments of the second aspect, the threshold percentage is about 40%, and if the pressure acceleration exceeds the threshold range by about 40%, the output command indicates an oncoming cough.

In some embodiments of the second aspect, the air pulse generator is fluidly connected to the garment with one or more hoses. In some embodiments of the second aspect, the air pulse generator, the hoses, and the garment form a closed system.

In some embodiments of the second aspect, the method further comprises automatically pausing the air pulse generator to allow the patient to cough if the output command is indicative of an oncoming cough.

According to a third aspect of a the present disclosure, an air pulse generator comprises an air chamber, a sensor positioned in pneumatic communication with the air chamber and configured to measure pressure in the air chamber, and a controller coupled to the sensor, the controller including a processor and a memory device, the memory device including instructions that, when executed by the processor, cause the controller to receive a signal from the sensor to measure the pressure in the air chamber over time, evaluate the signal to determine a cough indicator and generate an output command to modify a therapy provided to a patient using the air pulse generator in response to the cough indicator.

In some embodiments of the third aspect, the controller is configured to receive a first signal from the sensor when the patient is inhaling and a second signal from the sensor when the patient is exhaling, analyze the first and the second signals to determine the cough indicator.

In some embodiments of the third aspect, when the cough indicator exceeds a threshold range by a threshold percentage, the controller is configured to generate the output command to indicate an oncoming cough.

In some embodiments of the third aspect, the air pulse generator is configured to be fluidly connected to a garment including a number of air sacs, and wherein the sensor is configured to measure pressure in the air sacs.

In some embodiments of the third aspect, the garment is configured to vibrate a patient chest when inflated, and wherein the output command is configured to automatically pause the vibration of the garment when the cough indicator exceeds the threshold range by the threshold percentage.

In some embodiments of the third aspect, the air pulse generator is fluidly connected to the garment with one or more hoses, and wherein the air pulse generator, the hoses, and the garment form a closed system.

In some embodiments of the third aspect, the cough indicator is a rate of change of pressure.

In some embodiments of the third aspect, the controller is configured to process the signal with a low pass filter and then differentiate the signal to calculate the rate of change of pressure.

In some embodiments of the third aspect, the controller is configured to calculate the rate of change of pressure for a fixed time interval to determine the threshold range.

In some embodiments of the third aspect, the fixed time interval is about 30 seconds.

In some embodiments of the third aspect, the threshold percentage is about 40%, and if the rate of change of pressure exceeds the threshold range by about 40%, the output command indicates an oncoming cough.

In some embodiments of the third aspect, the cough indicator is pressure acceleration.

In some embodiments of the third aspect, the controller is configured to process the signal with a low pass filter and then double differentiate the signal to calculate the pressure acceleration.

In some embodiments of the third aspect, the controller is configured to calculate the pressure acceleration for a fixed time interval to determine the threshold range.

In some embodiments of the third aspect, the fixed time interval is about 30 seconds.

In some embodiments of the third aspect, the threshold percentage is about 40%, and if the pressure acceleration exceeds the threshold range by about 40%, the output command indicates an oncoming cough.

In some embodiments of the third aspect, if the output command is indicative of an oncoming cough, the air pulse generator is configured to automatically pause to allow the patient to cough.

### BRIEF DESCRIPTION OF THE DRAWINGS

The detailed description particularly refers to the accompanying figures in which:

FIG. 1 illustrates a high-frequency chest wall oscillation (HFCWO) system including an inflatable garment;

FIG. 2 illustrates the mechanism of coughing and the effect of an oncoming cough on pressure changes in the HFCWO system;

FIG. 3 illustrates a cough monitoring system configured to communicate over a network with an output device and with the HFCWO system;

FIG. 4 illustrates one embodiment of the cough monitoring system;

FIG. 5 illustrates one embodiment of a cough detection protocol;

FIG. 6 illustrates an instance when the cough indicator exceeds the threshold value to predict an oncoming cough;

FIG. 7 illustrates the implementation of the cough detection protocol at 5 Hz;

FIG. 8 illustrates the implementation of the cough detection protocol at 20 Hz;

FIG. 9 illustrates an air pressure generator comprising a user interface and a display;

FIG. 10 illustrates a first display screen on the user interface referred to in FIG. 9 including a start therapy button that can be selected to initiate the operation of the air pressure generator;

FIG. 11 illustrates a second display screen on the user interface referred to in FIG. 9 showing a unlock button than can be selected to change the parameters of the therapy;

FIG. 12 illustrates a third display screen on the user interface referred to in FIG. 9 showing a therapy dial indicating the time remaining in the therapy;

FIG. 13 illustrates a fourth display screen on the user interface referred to in FIG. 9 showing the buttons available when the therapy is paused;

FIG. 14 illustrates a fifth display screen on the user interface referred to in FIG. 9 requiring confirmation when a user chooses to stop therapy;

FIG. 15 illustrates a sixth display screen on the user interface referred to in FIG. 9 showing a summary of the therapy after the therapy has been stopped;

FIG. 16 illustrates a seventh display screen on the user interface referred to in FIG. 9 showing options for altering therapy parameters when the user chooses to edit therapy;

FIG. 17 illustrates an eight display screen on the user interface referred to in FIG. 9 showing buttons that can be used to change the frequency of the therapy;

FIG. 18 illustrates a ninth display screen on the user interface referred to in FIG. 9 showing buttons that can be used to change the intensity of the therapy; and

FIG. 19 illustrates a tenth display screen on the user interface referred to in FIG. 9 showing buttons that can be used to change the duration of the therapy.

### DETAILED DESCRIPTION

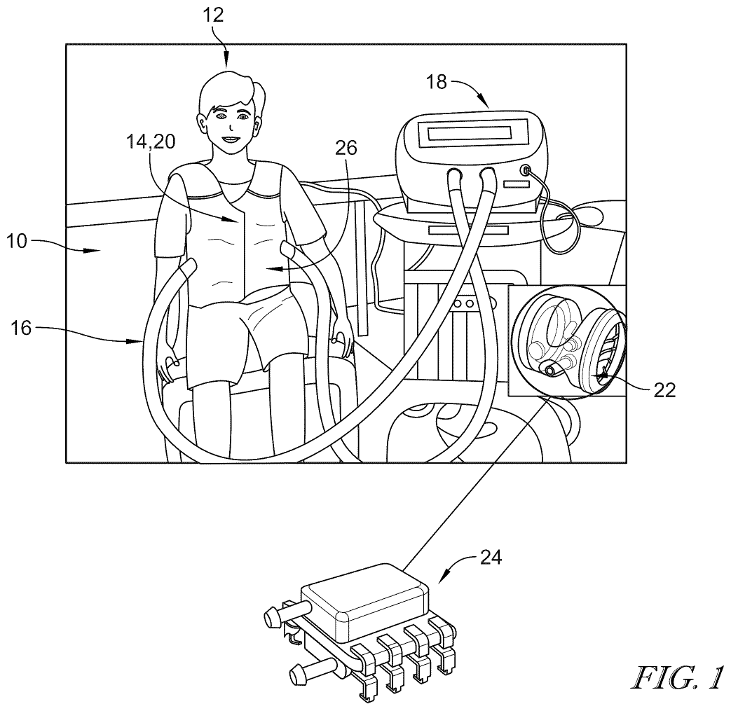

FIG. 1 illustrates a high-frequency chest wall oscillation (HFCWO) system 10. FIG. 1 shows a patient 12 using the HFCWO system 10. The HFCWO system 10 includes an inflatable garment 14, hoses 16, and an air pulse generator or air plethysmograph device 18. The garment 14 may be a vest 14 or any other body covering. The garment 14 is positioned on the chest of the patient 12. The one or more hoses 16 are configured to fluidly connect the garment 14 and the air pulse generator 18.

The hoses 16 connect an air chamber 22 in the air pulse generator 18 to one or more air sacs 20 in the garment 14. When operational, the air pulse generator 18 including the air chamber 22 can provide air pulses and a bias pressure to the garment 14. The one or more air sacs 20 in the garment 14 can be filled with air generated by the air pulse generator 18. The air pulses can vibrate or oscillate the garment 14, while the bias pressure can keep the garment 14 inflated. The garment 14 can apply an oscillating or vibrating compressive force to the chest of the patient 12. The system 10 can produce high-frequency chest wall oscillations or vibrations though the garment 14. The oscillating or vibrating compressive force may clear mucous and/or induce deep sputum from the lungs of the patient 12.

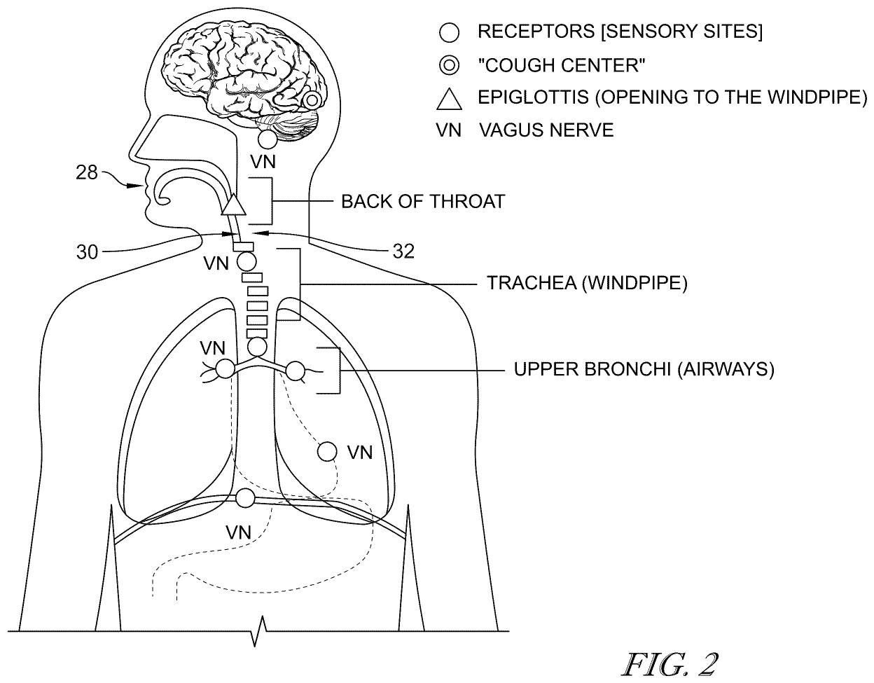

The mechanism associated with coughing and the effect of an oncoming cough on pressure changes in the system 10 is illustrated in FIG. 2. When the patient 12 inhales a volume of air as seen in step 28, pressure on the garment 14 may increase due to the patient's ribcage (or chest) movement. When the patient 12 is breathing in, his ribcage or chest may expand, compressing the garment 14. As shown in step 30, the opening to the trachea (the epiglottis) closes as the chest of the patient 12 constricts. The closing of the epiglottis may compress the air within the lungs of the patient 12. As shown in step 32, when the epiglottis opens, a rapid burst of air is allowed to be expelled through the mouth of the patient 12. When the patient 12 is breathing out, his ribcage or chest may collapse, causing a sudden pressure drop, and reducing the compression on the garment 14. Therefore, as illustrated, the physical movement of a patient's ribcage or chest results in an associated change in pressure on the garment 14 and the associated system 10.

As shown in FIG. 1, the garment 14, the hoses 16, and the air chamber 22 positioned in the air pulse generator 18 can form an enclosure 26. The enclosure 26 may be used for measuring pressure changes when the patient 12 breathes during therapy. In some embodiments, the enclosure 26 can be a closed system such that the ideal gas Boyle' law can be applied to the enclosure 26. Any change in volume of air in the air sacs 20 in the garment 14 due to ribcage compression can change the pressure in the enclosure 26. In some embodiments, the enclosure 26 comprising the garment 14, the hoses 16, and the air chamber 22 can have minimal leakage. In some embodiments, even though the enclosure 26 may not be a completely closed system, Boyle's law may still be applied to the enclosure 26 if the enclosure 26 has minimal leakage.

In some embodiments, the pressure changes may be measured by a sensor 24 (e.g., pressure sensor, flow sensor, etc.) positioned in the air chamber 22 in the air pulse generator 18. In other embodiments, the sensor 24 may be positioned at a different location in the enclosure 26. The sensor 24 may be a single-point pressure sensor 24 or may be a differential pressure sensor 24.

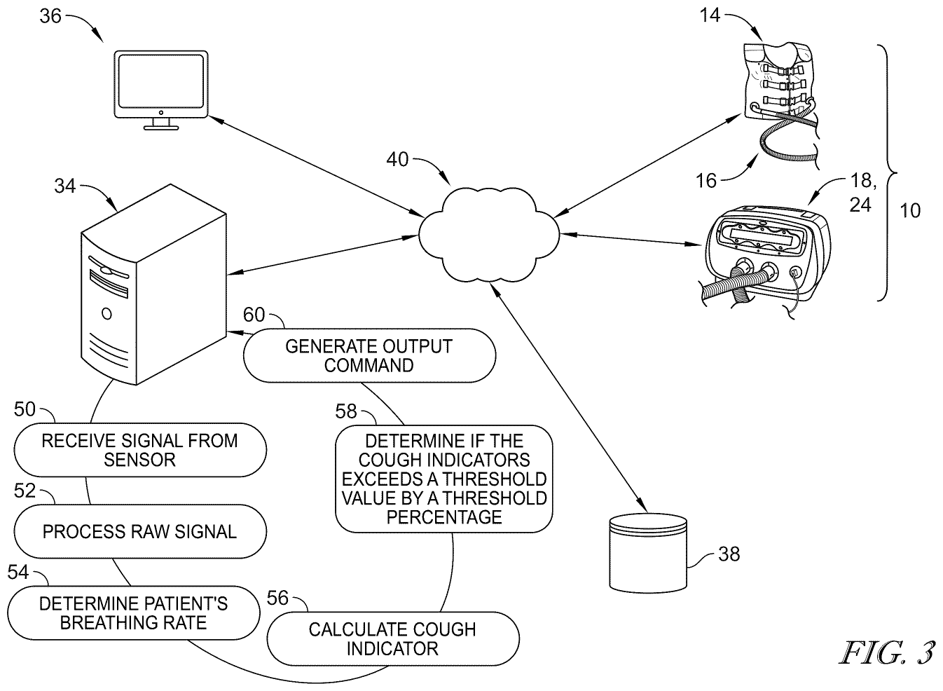

FIG. 3 shows a cough monitoring system 34 configured to communicate over a network 40 with an output device 36 and with the HFCWO system 10 comprising the garment 14, the hoses 16, and the air pulse generator 18. The cough monitoring system 34 and the HFCWO system 10 may communicate through manual entry, wired, wireless and/or power line connections and associated protocols (e.g., Ethernet, InfiniBand®, Bluetooth®, Wi-Fi®, WiMAX, 3G, 4G LTE, 5G, etc.). The cough monitoring system 34 may communicate with an external data storage devices or electronic medical record systems 38 over the network 40. The cough monitoring system 34 may display changing pressure measurements as discussed in FIG. 2 on the output device 36. The cough monitoring system 34 may transmit the information wirelessly to a remote location or output device 36 through the network 40. Alternatively, or additionally, the cough monitoring system 34 may transmit the information through a wired connection to one or more output devices 36.

The cough monitoring system 34 is configured to utilize data and, when appropriate, instruct the HFCWO system 10 to provide an output command indicating that the patient 12 has an oncoming cough. The output command may include a visual and/or audio signal. The output command may be a change in color of a light-emitting diode (LED) or may be a written indicator. Further, the output command may include a command to the HFCWO system 10 to pause the vibration of the inflatable garment 14 so that the patient 12 can cough.

The cough monitoring system 34 may be a control system or a controller embedded in a control system. The cough monitoring system 34 may be comprised in a device or an apparatus located in the patient vicinity. In some embodiments, the cough monitoring system 34 may be a computer or a mobile computing device. In other embodiments, the cough monitoring system 34 may be positioned in a room different than that of the patient 12 and may wirelessly communicate with the HFCWO system 10.

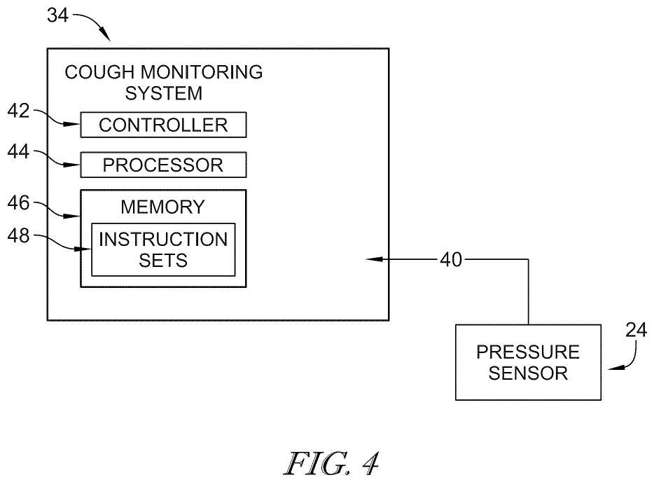

As shown in FIG. 4, the cough monitoring system 34 may include a processor 42 and a controller 44. The sensor 24 (e.g., pressure sensor, flow sensor, etc.) located in the air pulse generator 18 can communicate with the cough monitoring system 34 through the network 40. The sensor 24 (e.g., pressure sensor, flow sensor, etc.) and the cough monitoring system 34 can communicate through manual entry, wired, wireless and/or power line connections and associated protocols (e.g., Ethernet, InfiniBand®, Bluetooth®, Wi-Fi®, WiMAX, 3G, 4G LTE, 5G, etc.). The communication between the sensor 24 (e.g., pressure sensor, flow sensor, etc.) and the cough monitoring system 34 may also allow information regarding patient vital signs, including changes in pressure, to be transferred to other locations in the hospital or other facilities. The cough monitoring system 34 can comprise one or more instruction sets 48 stored in a memory device 46 and executed by one or more processors 42. The instruction set 48 may be utilized by the cough monitoring system 34 to determine or predict an oncoming cough by the patient using the HFCWO system 10.

The cough monitoring system 34 can determine detect, and/or predict an oncoming cough by executing a cough detection protocol 64. Briefly, as shown in FIG. 3, the cough monitoring system 34 can receive a signal from the sensor 24 (e.g., pressure sensor, flow sensor, etc.) positioned in the air chamber 22 of the air pulse generator 18 in step 50. In step 52, the processor 42 of the cough monitoring system 34 can process the raw signal based on the instruction set 48 stored in the memory device 46. In step 54, the cough monitoring system 34 can determine the patient's breathing rate. In step 56, the cough monitoring system 34 can calculate a cough indicator 62. In step 58, the cough monitoring system 34 can determine if the cough indicator 62 exceeds a threshold value by a threshold percentage. In step 60, the cough monitoring system 34 generates the output command.

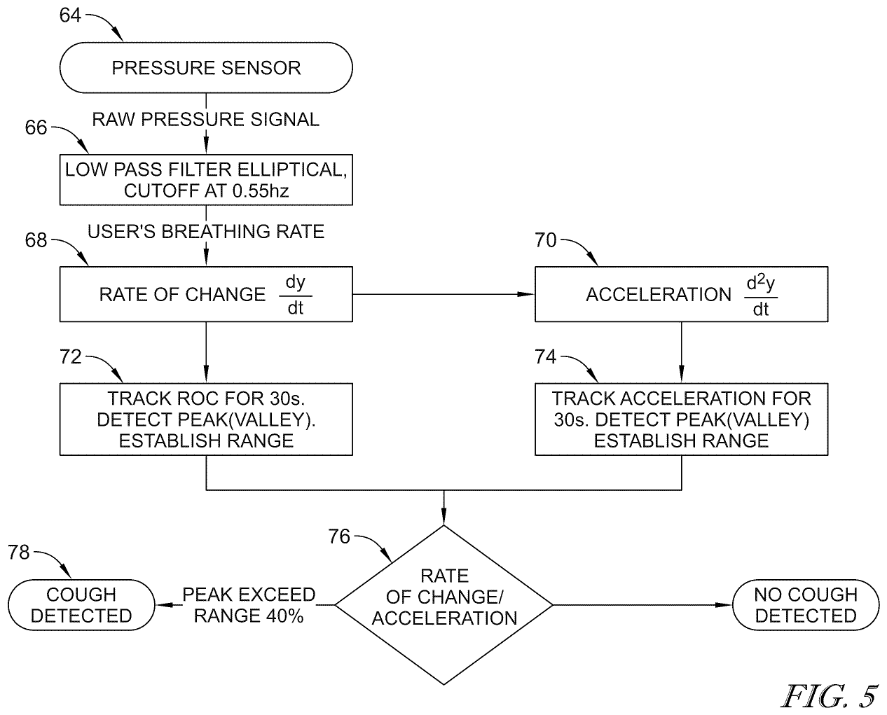

One embodiment of the cough detection protocol 64 is described in detail in FIG. 5. The cough monitoring system 34 is configured to receive a first signal from the sensor 24 (e.g., pressure sensor, flow sensor, etc.) when the patient 12 is inhaling and a second signal from the sensor 24 (e.g., pressure sensor, flow sensor, etc.) when the patient is exhaling in step 66. The cough monitoring system 34 can process the signals by passing the signals through a low pass filter with a cutoff at about 0.55 Hz. In some embodiments, the cut-off frequency may range from about 0.4 Hz to about 0.6 Hz, including any frequency or range comprised therein. In other embodiments, the cut-off frequency may be less than about 0.4 Hz. In other embodiments, the cut-off frequency may be more than about 0.6 Hz.

The cough monitoring system 34 can measure the patient's breathing rate and analyze the first and the second signals to determine the cough indicator 62 in steps 68 and 70. The cough indicator 62 can be a rate of change of pressure (measured in step 68) or pressure acceleration (measured in step 70). In steps 72 and 74, the cough monitoring system 34 can evaluate the cough indicator 62 over a certain time period to determine a threshold range of the cough indicator 62. For example, the cough monitoring system 34 can measure the rate of change of pressure over a certain time period of about 30 seconds to determine the threshold range of the rate of change of pressure (step 72). The threshold range may be an average rate of change of pressure as measured over the time period. The rate of change of pressure can be obtained by differentiating the pressure measurements. In some embodiments, this time period may range from about 20 seconds to about 40 seconds, about 40 seconds to about 60 seconds, about 60 seconds to about 80 seconds, about 80 seconds to about 100 seconds, or about 100 seconds to about 120 seconds including any time period or range comprised therein.

Similarly, the cough monitoring system 34 can measure the pressure acceleration over a time period of about 30 seconds to determine the threshold range of pressure acceleration (step 74). The rate of change of pressure can be obtained by differentiating the rate of change of pressure determination. In some embodiments, this time period may range from about 20 seconds to about 40 seconds, about 40 seconds to about 60 seconds, about 60 seconds to about 80 seconds, about 80 seconds to about 100 seconds, or about 100 seconds to about 120 seconds including any time period or range comprised therein.

During cough, ribcage transitions are generally fast and can cause the cough indicator 62 (rate of change of pressure or the pressure acceleration) to spike. The cough detection protocol 64 can identify the spikes and use the identification of such spikes to recognize or predict cough events (e.g., an oncoming cough). The cough monitoring system 34 can determine if the cough indicator 62 (rate of change of pressure or the pressure acceleration) exceeds the threshold range determined in step 72 or step 74 by a threshold percentage in step 76. If the cough monitoring system 34 determines that the cough indicator 62 (rate of change of pressure or the pressure acceleration) exceeds the threshold range determined in step 72 or step 74 by a threshold percentage, the cough monitoring system 34 can generate an output command indicating an oncoming cough in step 78. If the cough monitoring system 34 determines that the cough indicator 62 (rate of change of pressure or the pressure acceleration) does not exceed the threshold range determined in step 72 or step 74 by a threshold percentage, the cough monitoring system 34 does not generate an output command indicating an oncoming cough. If the cough monitoring system 34 determines that the cough indicator 62 (rate of change of pressure or the pressure acceleration) exceeds the threshold range determined in step 72 or step 74 by a threshold percentage, the cough monitoring system 34 can automatically pause the therapy for a cough pause.

The threshold percentage can range from about 30% to about 40%, from 40% to about 50%, or from about 25% to about 45% more than the threshold range, including any percentage or range comprised therein. In some embodiments, the threshold percentage may be about 40% more than the threshold range. In some embodiments, the output command is configured to automatically without patient intervention pause the vibration of the garment 14 when the cough indicator 62 exceeds the threshold range by the threshold percentage. In some embodiments, the output command is configured to pause the vibration of the garment 14 with minimal patient intervention when the cough indicator 62 exceeds the threshold range by the threshold percentage.

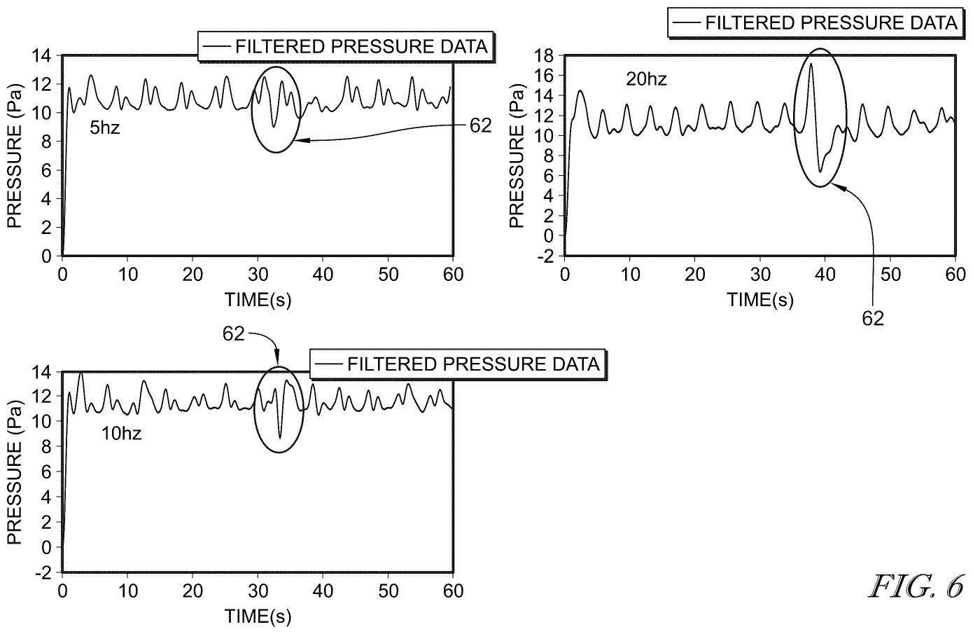

FIG. 6 illustrates an instance when the cough indicator 62 exceeds the threshold value. The cough indicator 62 shown in FIG. 6 is the rate of change (ROC) of pressure which is tracked by differentiating the pressure signal acquired from the patient 12. A quick drop in pressure can result in high ROC. The cough indicator 62 is illustrated at different frequencies (5 Hz, 10 Hz, 20 Hz).

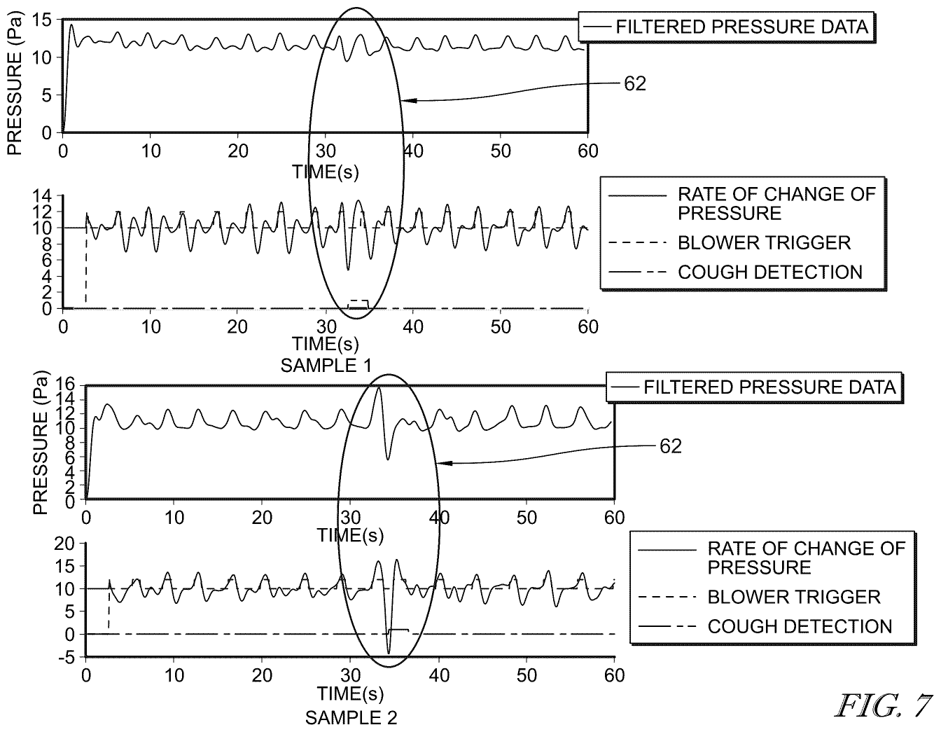

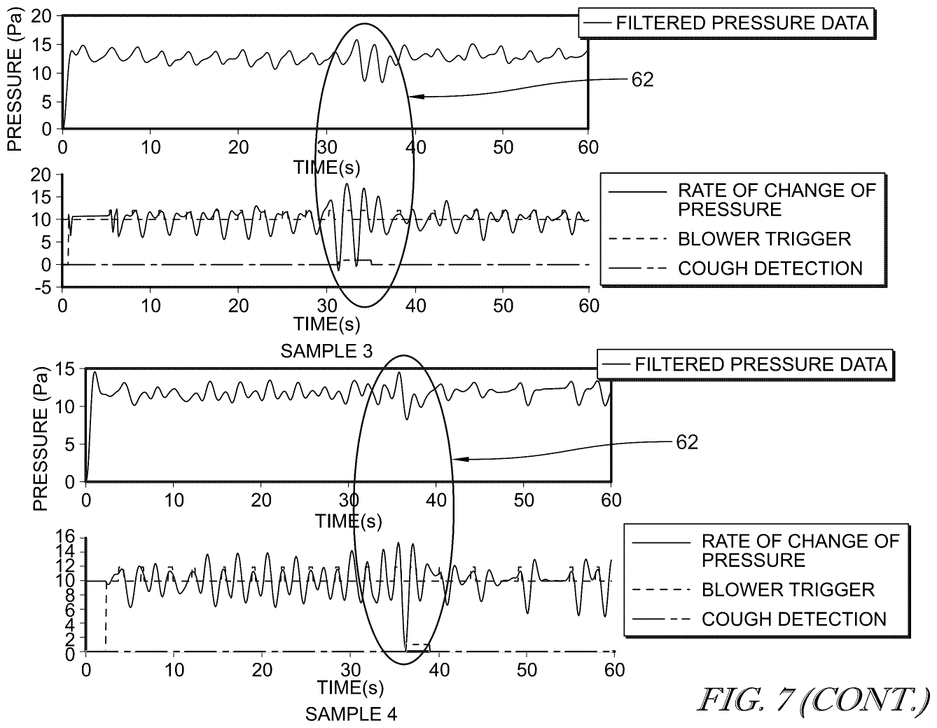

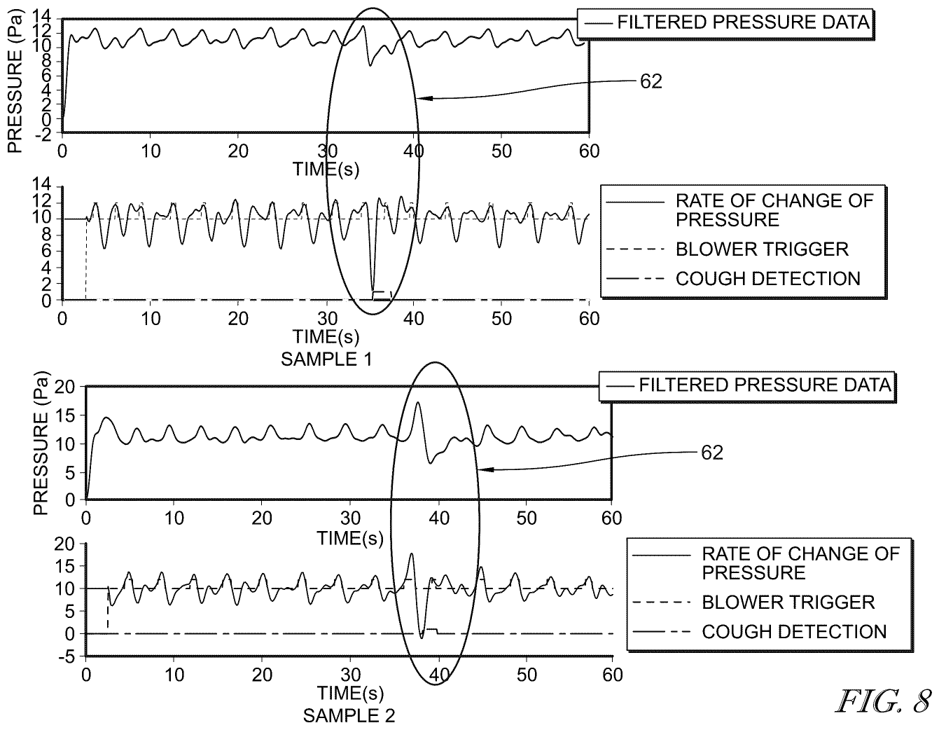

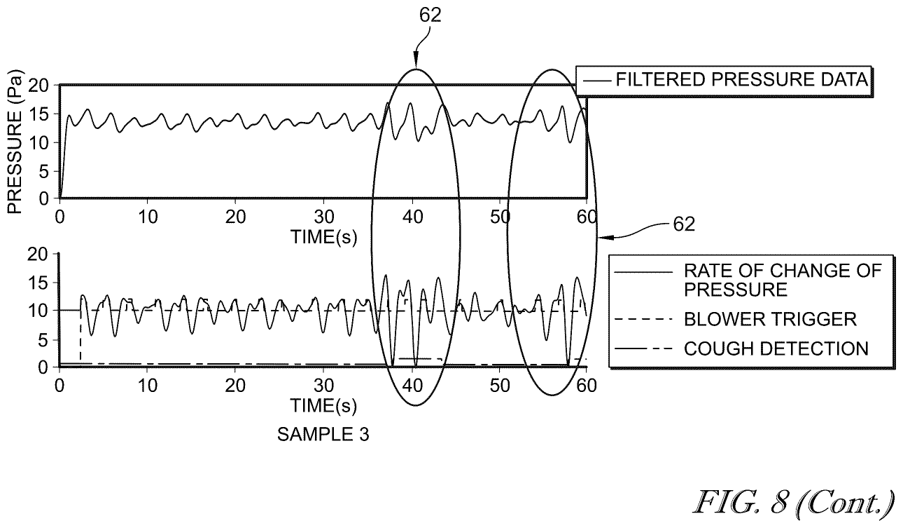

FIGS. 7 and 8 illustrate the implementation of the cough detection protocol 64 shown in FIG. 5. Volunteers were requested to cough after therapy of about 30 seconds and the cough indicator 62 was successfully detected prior to the occurrence of patient cough. Therefore, the cough indicator 62 was able to successfully predict an oncoming cough. FIG. 7 shows the rate of change of pressure at 5 Hz frequency and FIG. 8 shows the rate of change of pressure at 20 Hz frequency.

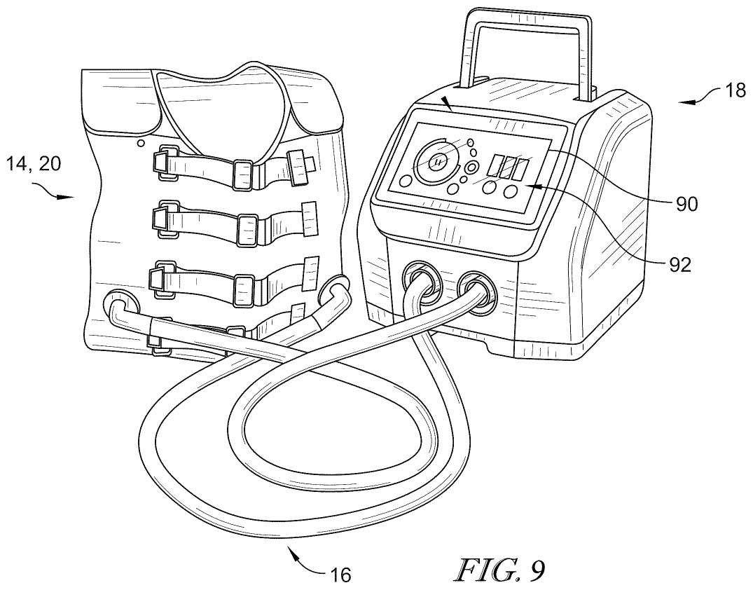

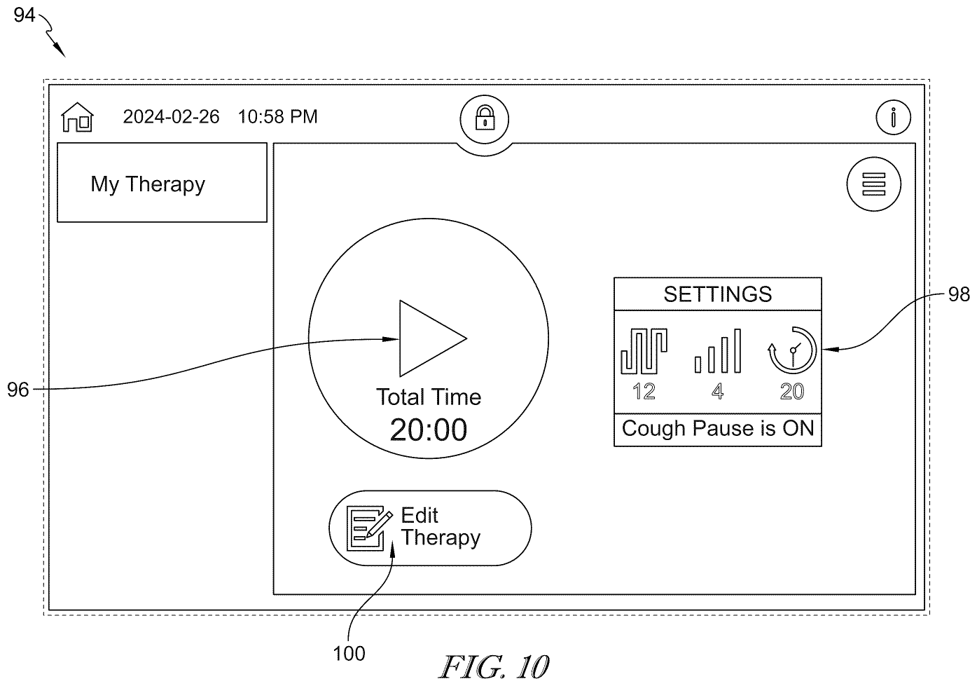

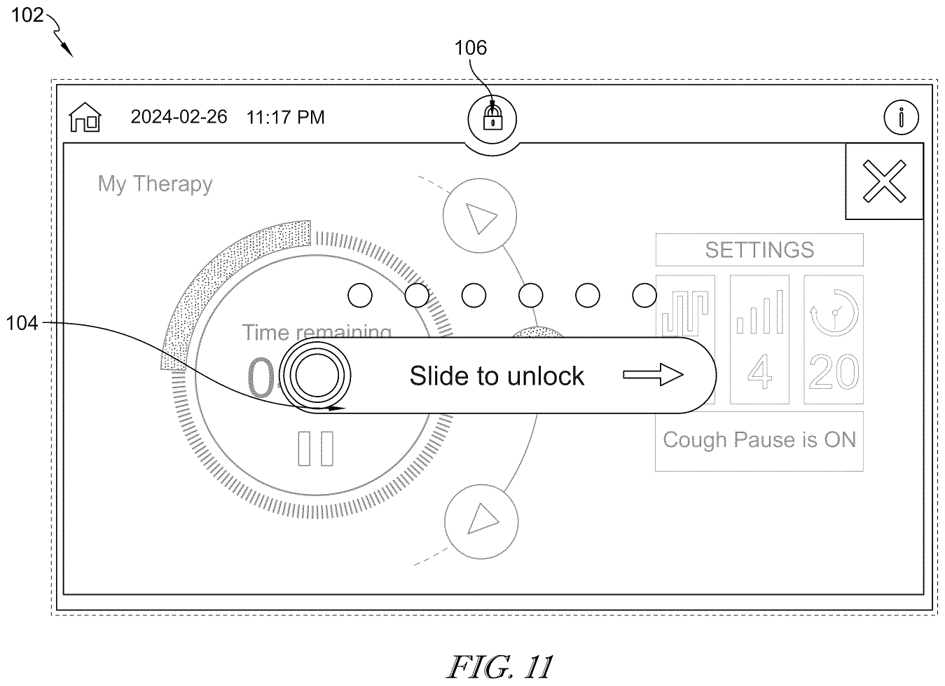

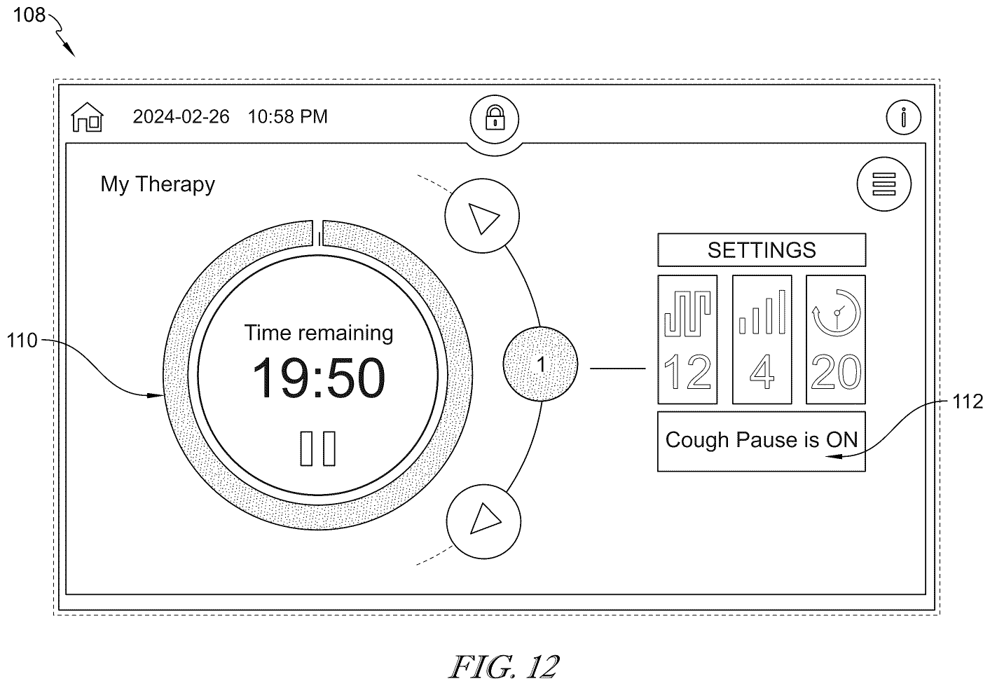

In some embodiments, as shown in FIG. 9, the output device 36 may be a user interface 90 including a display 92. The user interface 90 can be located on the air pressure generator 18. The user interface 90 can utilize data to illustrate different parameters in a therapy protocol being executed by the air pressure generator 18. In one embodiments, as shown in FIG. 10, the display 92 includes a first display screen 94. The first display screen 94 includes a start therapy button 96, which when selected by a user begins operating the air pressure generator based on the therapy parameters shown in a therapy panel 98. The first display screen 94 also includes an edit therapy button 100 that can be selected to change the parameters of the therapy being executed by the air pressure generator 18. When the edit therapy button 100 is selected, a second display screen 102 is configured to appear as shown in FIG. 11. The second display screen 102 includes a unlock button 104 that can be selected by the user to unlock the settings to edit the therapy parameters. The lock button 106 is configured to indicate with the settings are locked or unlocked. When the start therapy button 96 shown in the first display screen 94 is selected the air pressure generator 18 is turned on and the time remaining in the therapy is indicated on a therapy dial 110 as shown in a third therapy screen 108 as shown in FIG. 12.

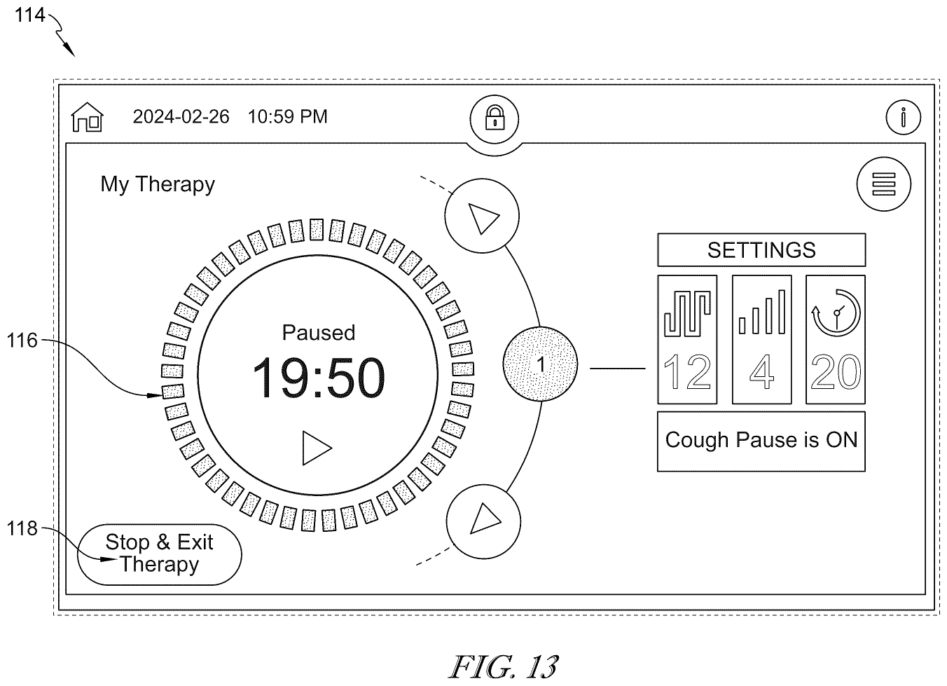

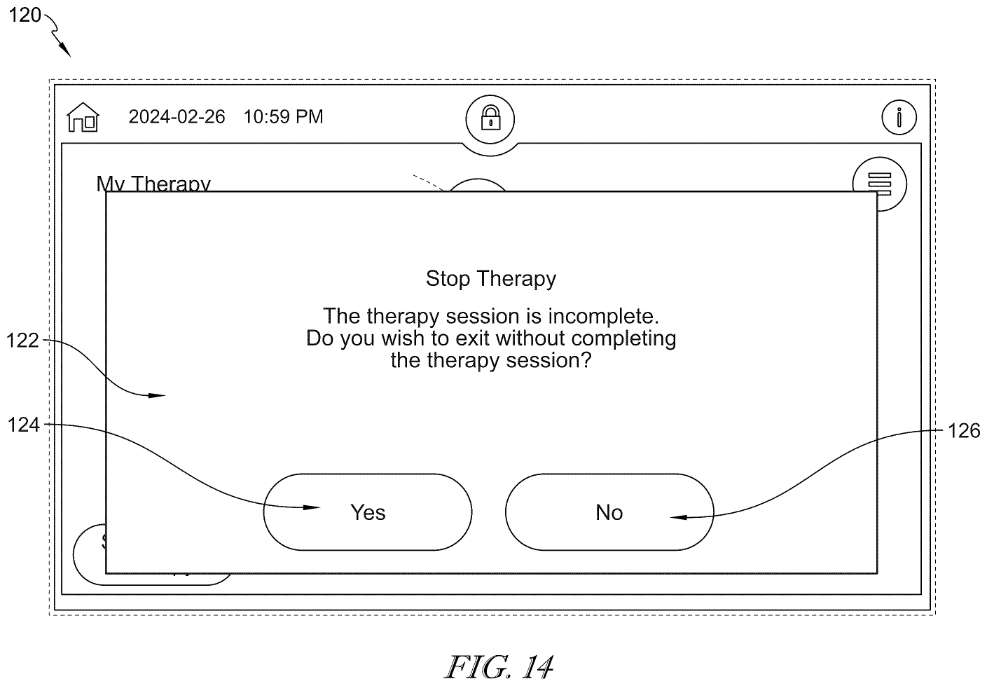

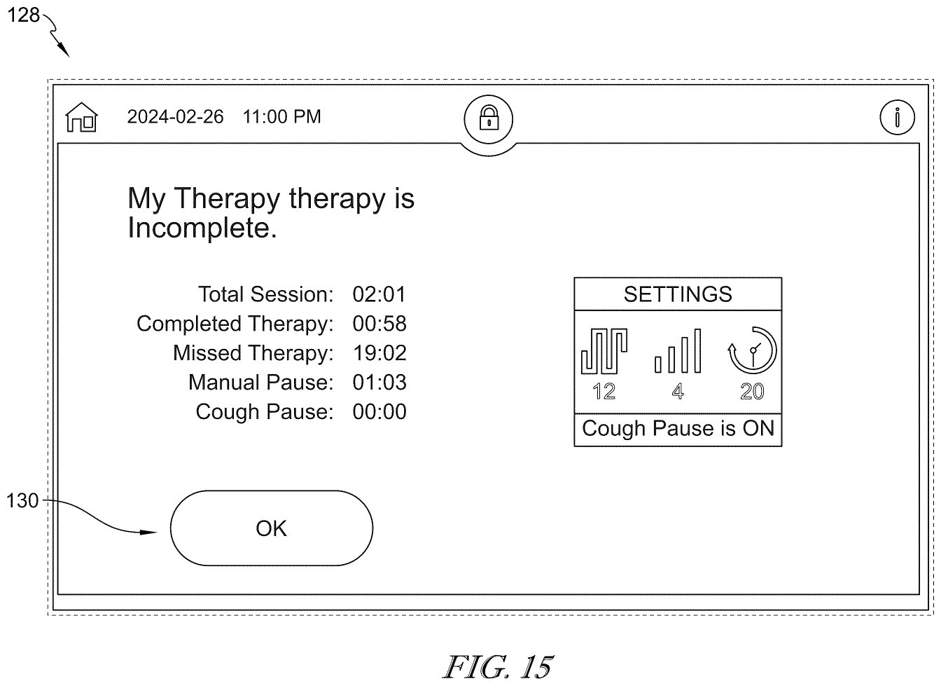

The ongoing therapy provided by the air pressure generator can either be paused manually by selecting the therapy dial 110 or automatically when an oncoming cough is detected based on the cough detection protocol 64 described in FIG. 5. Panel 112 indicates when the cough pause based on the algorithm described above is switched on. If an oncoming cough is detected or if therapy is manually paused, a fourth display screen 114 is configured to appear as shown in FIG. 13. A dial 116 indicates the amount of time that the therapy is paused. The user can restart therapy by selecting the dial 116. Alternatively, the user can stop and exit therapy by selecting the stop therapy button 118. If the user selects the stop therapy button 118, a fifth display screen 120 is configured to appear as shown in FIG. 14. The fifth display screen 120 includes a confirmation screen 122 with a yes button 124 and a no button 126. Selection of the yes button 124 ends therapy and selection of the no button 126 reverts to the fourth display screen 118 shown in FIG. 13. Selection of the yes button 124 causes a sixth therapy screen 128 to appear as shown in FIG. 15. The sixth display screen 128 summarizes the therapy on a summary screen 130 that also lists the amount of time paused due to an oncoming cough detected based on the cough detection protocol 64 described in FIG. 5 and/or due to any manual pauses initiated by the user.

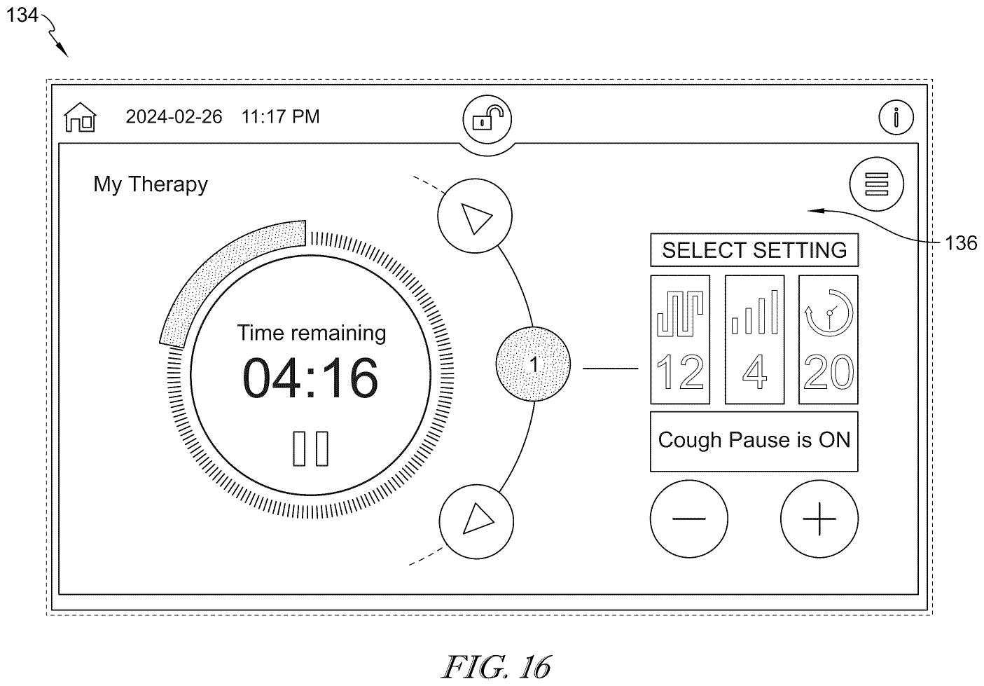

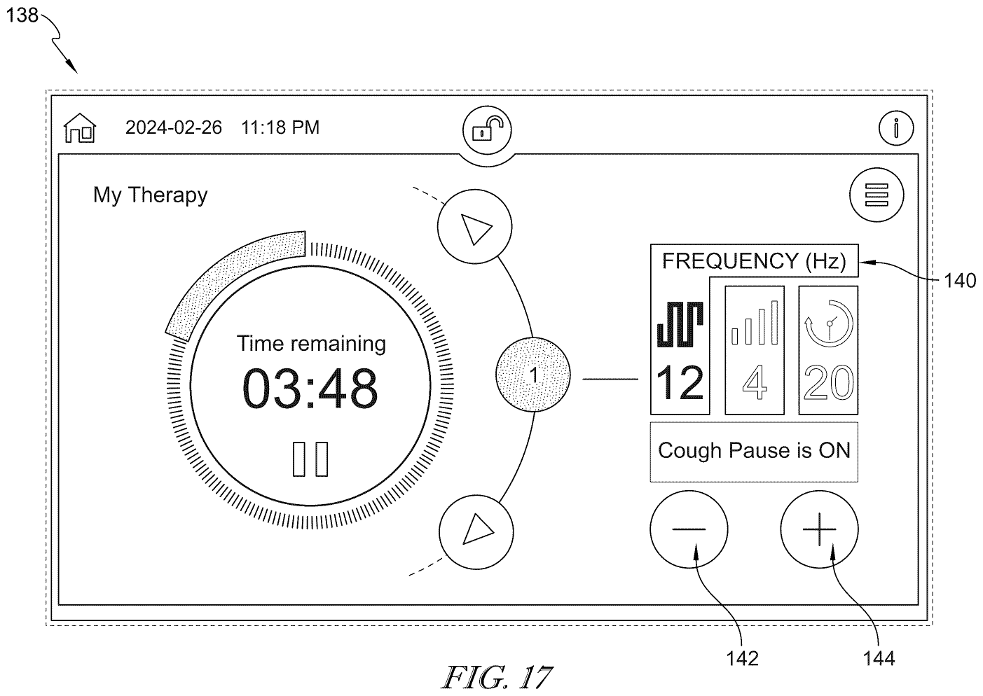

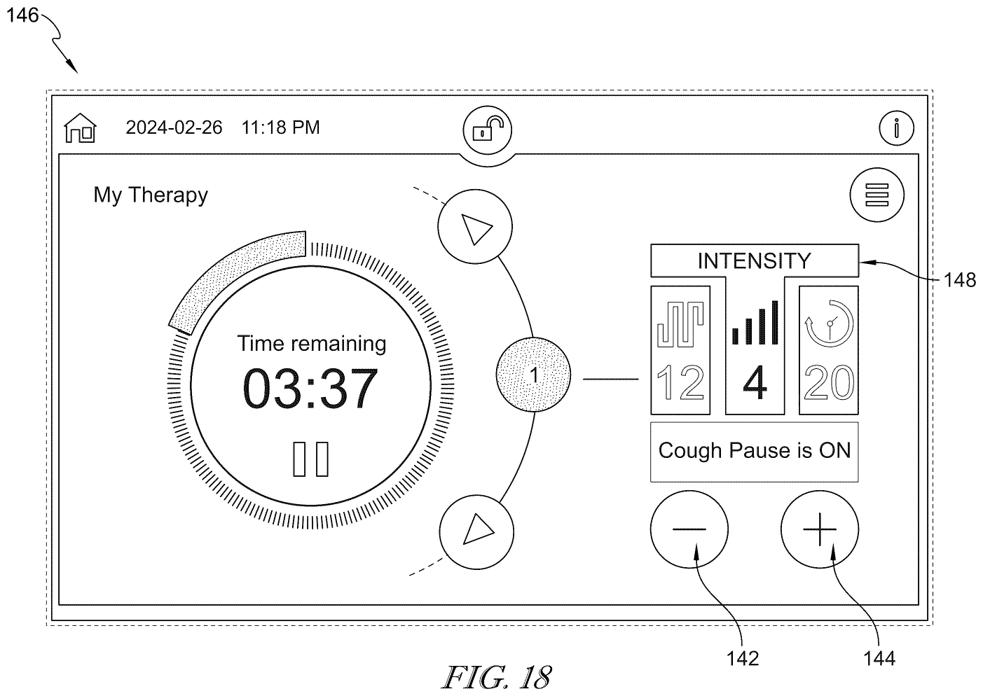

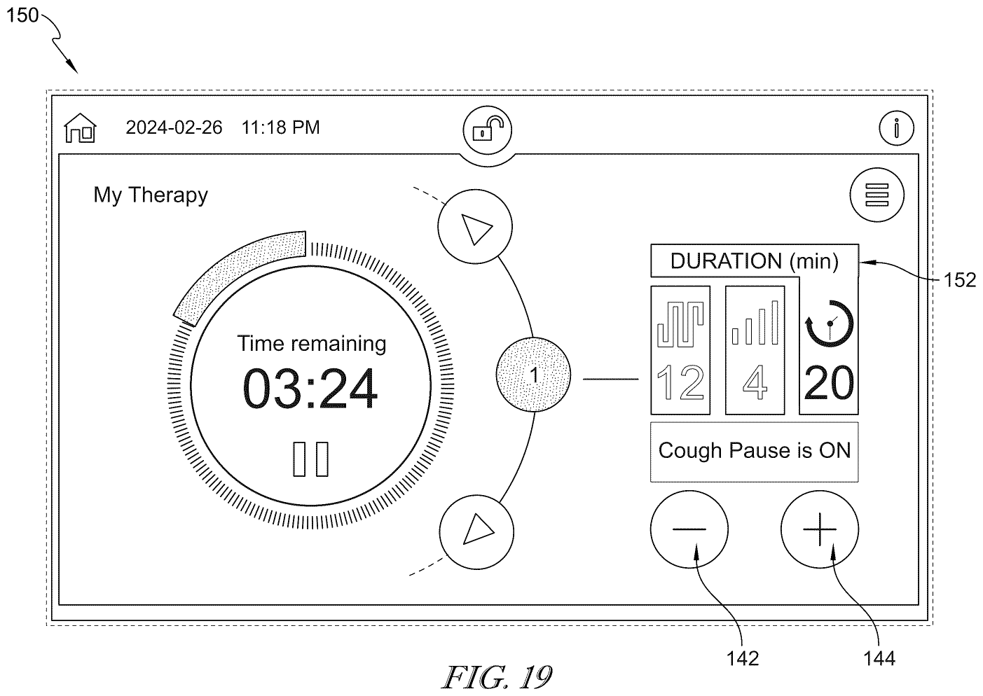

When the unlock button 104 shown on the second display screen 102 is selected by the user to unlock the settings to edit the therapy parameters, a seventh display screen 134 configured to appear as shown in FIG. 16. A selection panel 136 can be used to select the therapy parameter that the user wants to change. As shown in display screens 138, 146, 150, the user can select to change frequency 140, intensity 148, or duration 152 (FIGS. 17-19). An up button 144 and a down button 142 can be used to change the values of frequency 140 (FIG. 17), intensity 148 (FIG. 18), or duration 152 (FIG. 19) of the therapy setting. Therapy can be restarted after the settings are changed.

Although this disclosure refers to specific embodiments, it will be understood by those skilled in the art that various changes in form and detail can be made without departing from the subject matter set forth in the accompanying claims.

1. A chest wall oscillation therapy apparatus comprising:
a garment including a plurality of air sacs;
an air pulse generator including an air chamber fluidly connected to the garment and a sensor, wherein rapid mechanical compression of air in the air chamber causes an inflation of the plurality of air sacs, and wherein the sensor is positioned in pneumatic communication with the air chamber and configured to measure pressure in the air chamber, and
a controller coupled to the sensor, the controller including a processor and a memory device, the memory device including instructions that, when executed by the processor, causes the controller to receive a signal from the sensor to measure the pressure in the air chamber over time, analyze the signal to determine a cough indicator, and generate an output command to modify a therapy provided to a patient wearing the garment in response to the cough indicator.

2. The apparatus of claim 1, wherein the controller is configured to receive a first signal from the sensor when the patient is inhaling and a second signal from the sensor when the patient is exhaling, analyze the first signal and the second signal to determine the cough indicator.

3. The apparatus of claim 2, wherein if the cough indicator exceeds a threshold range by a threshold percentage, the controller is configured to generate the output command to indicate an oncoming cough.

4. The apparatus of claim 3, wherein the garment is configured to cause a vibration of a patient chest when inflated, and wherein the output command is configured to automatically pause the vibration when the cough indicator exceeds the threshold range by the threshold percentage.

5. The apparatus of claim 2, wherein the air pulse generator is fluidly connected to the garment with one or more hoses, and wherein the air pulse generator, the one or more hoses, and the garment are configured to form a closed system.

6. The apparatus of claim 3, wherein the cough indicator is a rate of change of pressure.

7. The apparatus of claim 3, wherein the cough indicator is pressure acceleration.

8. A method of diagnosing an oncoming cough in a patient using a chest wall oscillation therapy apparatus, comprising:
wearing a garment including a plurality of air sacs;
inflating the garment by using an air pulse generator including an air chamber fluidly connected to the garment;
receiving a signal from the garment, the signal indicative of pressure in the garment and the air chamber over a period of time by using a sensor positioned in the air pulse generator;
processing the signal to determine a cough indicator;
diagnosing the oncoming cough if the cough indicator exceeds a threshold range by a threshold percentage;
generating an output command to indicate the oncoming cough; and
modifying therapy to the patient wearing the garment in response to the cough indicator.

9. The method of claim 8, wherein the method comprises receiving a first signal indicative of a first pressure when inhaling air and receiving a second signal indicative of a second pressure when expelling air.

10. The method of claim 8, wherein the method comprises the garment vibrating a patient chest after inflating the garment.

11. The method of claim 10, wherein the method comprises generating the output command to automatically pause the vibration of the garment when the cough indicator exceeds the threshold range by the threshold percentage.

12. The method of claim 8, wherein the cough indicator is a rate of change of pressure.

13. The method of claim 12, wherein the method comprises processing the signal with a low pass filter and then differentiating the signal to calculate the rate of change of pressure.

14. The method of claim 8, wherein the cough indicator is pressure acceleration.

15. The method of claim 14, wherein the method comprises processing the signal with a low pass filter and then double differentiating the signal to calculate the pressure acceleration.

16. An air pulse generator comprising:
an air chamber,
a sensor positioned in pneumatic communication with the air chamber and configured to measure pressure in the air chamber, and
a controller coupled to the sensor, the controller including a processor and a memory device, the memory device including instructions that, when executed by the processor, cause the controller to receive a signal from the sensor to measure the pressure in the air chamber over time, analyze the signal to determine a cough indicator and generate an output command to modify a therapy provided to a patient using the air pulse generator in response to the cough indicator.

17. The air pulse generator of claim 16, wherein the controller is configured to receive a first signal from the sensor when the patient is inhaling and a second signal from the sensor when the patient is exhaling, analyze the first signal and the second signal to determine the cough indicator.

18. The air pulse generator of claim 17, wherein if the cough indicator exceeds a threshold range by a threshold percentage, the controller is configured to generate the output command to indicate an oncoming cough.

19. The air pulse generator of claim 16, wherein the air pulse generator is configured to be fluidly connected to a garment including a plurality of air sacs, and wherein the sensor is configured to measure a pressure in the plurality of air sacs.

20. The air pulse generator of claim 19, wherein the garment is configured to vibrate a patient chest when inflated, and wherein the output command is configured to automatically pause the vibration of the garment when the cough indicator exceeds a threshold range by a threshold percentage.
